(19) United States
(12) Patent Application Publication
(10) Pub. No.: US 2025/0003920 A1
(43) Pub. Date: Jan. 2, 2025

(54) SENSOR ELEMENT

(71) Applicant: NGK INSULATORS, LTD., Nagoya-shi (JP)

(72) Inventors: Shiho IWAI, Nagoya-shi (JP); Sotaro INAGAKI, Komaki-shi (JP); Ryota TOMIOKA, Nagoya-shi (JP); Takayuki SEKIYA, Nisshin-shi (JP)

(21) Appl. No.: 18/883,280

(22) Filed: Sep. 12, 2024

Related U.S. Application Data
(63) Continuation-in-part of application No. PCT/JP2023/010918, filed on Mar. 20, 2023.

(30) Foreign Application Priority Data
Mar. 28, 2022 (JP) 2022-051848

(51) Int. Cl. G01N 27/407; G01N 33/00

(52) U.S. Cl. CPC G01N 27/4075; G01N 27/4071; G01N 33/0027

(57) ABSTRACT

A sensor element for detecting a target gas to be measured in a measurement-object gas includes: a base part in an elongated plate shape, including an oxygen-ion-conductive solid electrolyte layer; a measurement-object gas flow part formed from one end part in a longitudinal direction of the base part; an inner main pump electrode disposed on an inner surface of the measurement-object gas flow part; a porous coating layer covering at least an electrode end part of the inner main pump electrode closer to the one end part in the longitudinal direction of the base part; and a measurement electrode disposed at a position farther from the one end part in the longitudinal direction of the base part than the inner main pump electrode on the inner surface of the measurement-object gas flow part.

### CROSS-REFERENCE TO RELATED APPLICATION

This application is a continuation application of PCT/JP2023/010918, filed on Mar. 20, 2023, which claims priority from Japanese Patent Application No. 2022-051848, filed on Mar. 28, 2022, the entire contents of which are incorporated herein by reference.

### BACKGROUND OF THE INVENTION

### Technical Field of the Invention

The present invention relates to a sensor element using an oxygen ion conductive solid electrolyte.

### Background Art

A gas sensor is used for detection or measurement of concentration of an objective gas component (oxygen O₂, nitrogen oxide NOx, ammonia NH₃, hydrocarbon HC, carbon dioxide CO₂, etc.) in a measurement-object gas, such as exhaust gas of automobile. For example, conventionally, the concentration of the objective gas component in exhaust gas of an automobile is measured, and an exhaust gas cleaning system mounted on the automobile is optimally controlled based on the measurement.

As such a gas sensor, a gas sensor equipped with a sensor element using an oxygen ion conductive solid electrolyte such as zirconia (ZrO₂) is known. The gas sensor detects an electromotive force or a current value corresponding to the concentration of an objective gas component in a measurement-object gas by using the oxygen ion conductivity of the solid electrolyte, thereby detecting the gas component and measuring the concentration.

For example, JP3050781B2 discloses a gas sensor that controls the oxygen partial pressure to such a low level that does not substantially affect measurement of the amount of a measurement-object gas component by means of a first electrochemical pumping cell and a second electrochemical pumping cell, and detects a current value corresponding to the oxygen generated by reduction or decomposition of the measurement-object gas component. In other words, oxygen is preliminarily removed by the first electrochemical pumping cell and the second electrochemical pumping cell, and the oxygen derived from the objective gas component (for example, nitrogen oxide NOx) is detected.

JP2020-101476A and JP2021-156611A disclose a NOx sensor equipped with a sensor element having a main pump cell and an auxiliary pump cell for adjusting oxygen concentration, and a measuring pump cell for detecting NOx in the measurement-object gas whose oxygen concentration has been adjusted.

In such a NOx sensor, it is known that Pt with Au added is used as a metal material in an inner pump electrode that constitutes the main pump cell and an inner auxiliary pump electrode that constitutes the auxiliary pump cell, so as not to decompose NOx (for example, in JP2021-156611A)

### CITATION LIST

### Patent Documents

Patent Document 1: JP3050781B2
Patent Document 2: JP2020-101476A
Patent Document 3: JP2021-156611A

### SUMMARY OF THE INVENTION

### Problems to be Solved by the Invention

Recently, there is a demand for further prolonging a service life of a gas sensor. For example, it was conventionally considered that a NOx sensor should maintain a specified performance for 3000 hours in a specified durability test using a diesel engine. Currently, however, significant further prolongation of a service life, for example, up to about 20,000 hours, is required.

The present inventors therefore diligently studied about the mechanism of decrease in detection accuracy of a gas sensor such as a conventional NOx sensor, and considered as follows. The decrease in the detection accuracy of the NOx sensor is considered to be caused due to (1) decrease in NOx decomposition activity in a measurement electrode that constitutes a measurement pump cell, and (2) NOx decomposition in an inner main pump electrode that constitutes a main pump cell. (1) The decrease in NOx decomposition activity in the measurement electrode is considered to be mainly caused by attachment of Au contained in the inner main pump electrode and an inner auxiliary pump electrode to the measurement electrode. Further, the present inventors focused on deterioration of the inner main pump electrode as a factor causing (2) the NOx decomposition in the inner main pump electrode. The deterioration of the inner main pump electrode is considered to be affected by (i) magnitude of a current flowing through the main pump cell, and (ii) temperature of the main pump electrode. For example, JP2021-156611A discloses that decomposition of NOx in the main pump cell is desirably suppressed, if current density of a current flowing though the main pump cell is 0.4 mA/mm² or less (claim 3). However, in JP2021-156611A, the current density represents average current density of the inner main pump electrode.

To achieve further prolongation of the service life as described above, it is necessary to further reduce the decrease in NOx decomposition activity in the measurement electrode and the NOx decomposition in the main pump cell, and to maintain high detection accuracy over a longer term use.

In light of this, it is an object of the present invention to provide a sensor element capable of suppressing decrease in detection accuracy due to long-term use of a gas sensor.

### Means for Solving the Problems

The present inventors further diligently studied, and found that current density distribution exists in an inner main pump electrode. A measurement-object gas is introduced through a gas inlet on one end part in a longitudinal direction of a sensor element, and reaches the inner main pump electrode. Especially when oxygen concentration in the measurement-object gas is high, a position closer to the gas inlet in the inner main pump electrode is exposed to oxygen of higher concentration, and thus more oxygen is pumped out. That is, current density of the current flowing in the main pump cell becomes larger, especially at the position closer to the gas inlet in the inner main pump electrode. Further, resistance of the main pump cell tends to increase as the gas sensor is used over a long period of time. As a result of these, NOx in the measurement-object gas may be decomposed, especially at the position close to the gas inlet in the inner main pump electrode. It is found that NOx decomposition in the inner main pump electrode can be further suppressed by mitigating current concentration at the position close to the gas inlet in the inner main pump electrode.

As a result of intensive studies, the present inventors found that by providing a porous coating layer that covers at least a part including an electrode end part of the inner main pump electrode closer to the one end part in the longitudinal direction of the sensor element, it is possible to suppress evaporation of Au from the inner main pump electrode, and to mitigate the current concentration at the position close to the gas inlet in the inner main pump electrode. As a result, the present inventors found that decrease in the detection accuracy due to long-term use of the gas sensor can be suppressed.

The present invention includes the following aspects.
(1) A sensor element for detecting a target gas to be measured in a measurement-object gas, the sensor element comprising:
   a base part in an elongated plate shape, including an oxygen-ion-conductive solid electrolyte layer;
   a measurement-object gas flow part formed from one end part in a longitudinal direction of the base part;
   an inner main pump electrode disposed on an inner surface of the measurement-object gas flow part;
   a porous coating layer covering at least an electrode end part of the inner main pump electrode closer to the one end part in the longitudinal direction of the base part; and
   a measurement electrode disposed at a position farther from the one end part in the longitudinal direction of the base part than the inner main pump electrode on the inner surface of the measurement-object gas flow part.
(2) The sensor element according to the above (1), wherein the porous coating layer covers 3% or more of area of the inner main pump electrode.
(3) The sensor element according to the above (1) or (2), wherein an oxygen diffusion coefficient of the porous coating layer is 1×10⁶ m²/s or less in at least a part of the porous coating layer.
(4) The sensor element according to any one of the above (1) to (3), wherein a thickness of the porous coating layer is 1 μm or more.
(5) The sensor element according to any one of the above (1) to (4), wherein an oxygen diffusion coefficient of the porous coating layer varies in the longitudinal direction of the base part.
(6) The sensor element according to any one of the above (1) to (5), wherein an oxygen diffusion coefficient of the porous coating layer is stepwise or continuously increased from a closer side toward a farther side with respect of the one end part in the longitudinal direction of the base part.

### Advantageous Effect of the Invention

According to the present invention, since evaporation of Au from the inner main pump electrode can be suppressed, attachment of Au to the measurement electrode can be suppressed. Further, according to the present invention, since the current concentration at the position close to the gas inlet in the inner main pump electrode can be mitigated, NOx decomposition in the inner main pump electrode can be further suppressed even when a resistance value of the main pump cell is increased due to long-term use of the gas sensor. As a result, the decrease in the detection accuracy due to the long-term use of the gas sensor can be suppressed.

### BRIEF DESCRIPTION OF THE DRAWINGS

FIG. 1 is a vertical sectional schematic view in a longitudinal direction of a sensor element 101, showing one example of a general configuration of a gas sensor 100.

FIG. 2 is a partial sectional schematic view showing an arrangement of respective electrodes 22, 51, and 44, and a porous coating layer 25 disposed in a measurement-object gas flow part 15. L_{E} indicates a length of an inner main pump electrode 22 in the longitudinal direction of the sensor element 101, and L_{C} indicates a length of a region of the inner main pump electrode 22 covered by the porous coating layer 25 in the longitudinal direction of the sensor element 101.

FIG. 3 is a sectional schematic view showing a part of the section along line III-III in FIG. 1. FIG. 3 is a schematic view showing a general planar arrangement of the inner main pump electrode 22, the porous coating layer 25, an auxiliary pump electrode 51, and a measurement electrode 44 disposed on an upper surface of a first solid electrolyte layer 4. L_{E} indicates the length of the inner main pump electrode 22 in the longitudinal direction of the sensor element 101, and L_{C} indicates the length of the region (a region illustrated by a dashed line in FIG. 3) of the inner main pump electrode 22 covered by the porous coating layer 25 in the longitudinal direction of the sensor element 101.

FIG. 4 is a schematic diagram showing the relation between oxygen concentration and a pump current Ip2 in the presence of oxygen (O₂=0, 5, 10, 18%).

FIG. 5 is a graph showing durability test results of Examples 1 to 2 and Comparative Example 1.

### MODES FOR CARRYING OUT OF THE INVENTION

A sensor element of the present invention includes:
a base part in an elongated plate shape, including an oxygen-ion-conductive solid electrolyte layer;
a measurement-object gas flow part formed from one end part in a longitudinal direction of the base part;
an inner main pump electrode disposed on an inner surface of the measurement-object gas flow part;
a porous coating layer covering at least an electrode end part of the inner main pump electrode closer to the one end part in the longitudinal direction of the base part; and
a measurement electrode disposed at a position farther from the one end part in the longitudinal direction of the base part than the inner main pump electrode on the inner surface of the measurement-object gas flow part.

### General Configuration of Gas Sensor

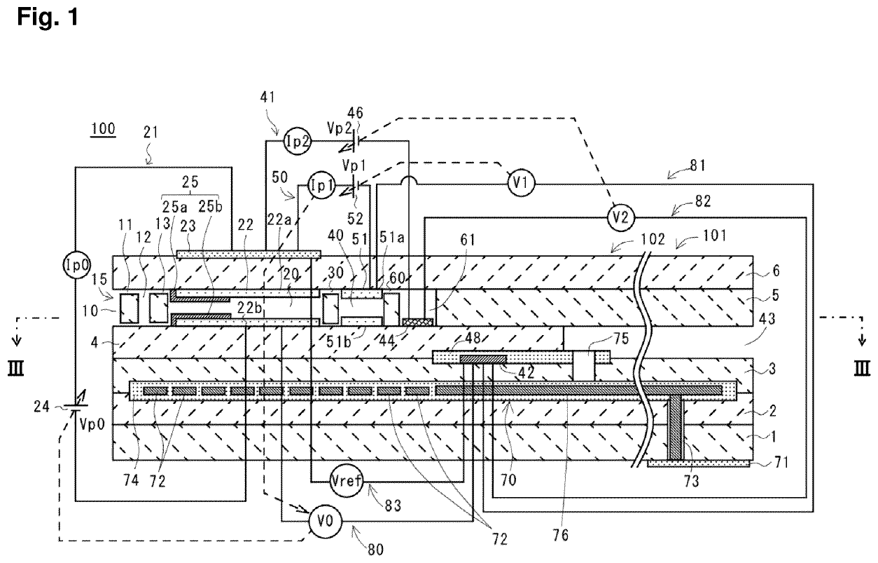

The sensor element of the present invention will now be described with reference to the drawings. FIG. 1 is a vertical sectional schematic view in the longitudinal direction, showing one example of a general configuration of a gas sensor 100 including a sensor element 101. Hereinafter, based on FIG. 1, the upper side and the lower side in FIG. 1 are respectively defined as top and bottom, and the left side and the right side in FIG. 1 are respectively defined as a front end side and a rear end side.

In the embodiment of FIG. 1, the gas sensor 100 represents one example of a limiting current type NOx sensor that detects NOx in a measurement-object gas by the sensor element 101, and measures the concentration of NOx.

The sensor element 101 is an element in an elongated plate shape, including a base part 102 having such a structure that a plurality of oxygen-ion-conductive solid electrolyte layers are layered. The elongated plate shape is also called a long plate shape or a belt shape. The base part 102 has such a structure that six layers, namely, a first substrate layer 1, a second substrate layer 2, a third substrate layer 3, a first solid electrolyte layer 4, a spacer layer 5, and a second solid electrolyte layer 6, are layered in this order from the bottom side, as viewed in the drawing. Each of the six layers is formed of an oxygen-ion-conductive solid electrolyte layer containing, for example, zirconia (ZrO₂). The solid electrolyte forming these six layers is dense and gastight. These six layers all may have the same thickness, or the thickness may vary among the layers. The layers are adhered to each other with an adhesive layer of a solid electrolyte interposed therebetween, and the base part 102 includes the adhesive layer. While a layer configuration composed of the six layers is illustrated in FIG. 1, the layer configuration in the present invention is not limited to this, and any number of layers and any layer configuration are possible.

The sensor element 101 is manufactured, for example, by stacking ceramic green sheets corresponding to the individual layers after conducting predetermined processing, printing of circuit pattern and the like, and then firing the stacked ceramic green sheets so that they are combined together.

A gas inlet 10 is formed between the lower surface of the second solid electrolyte layer 6 and the upper surface of the first solid electrolyte layer 4 in one end part in the longitudinal direction (hereinafter, referred to as a front end part) of the sensor element 101. A measurement-object gas flow part 15 is formed in such a form that a first diffusion-rate limiting part 11, a buffer space 12, a second diffusion-rate limiting part 13, a first internal cavity 20, a third diffusion-rate limiting part 30, a second internal cavity 40, a fourth diffusion-rate limiting part 60, and a third internal cavity 61 communicate in this order in the longitudinal direction from the gas inlet 10.

The gas inlet 10, the buffer space 12, the first internal cavity 20, the second internal cavity 40, and the third internal cavity 61 constitute internal spaces of the sensor element 101. Each of the internal spaces is provided in such a manner that a portion of the spacer layer 5 is hollowed out, and the top of each of the internal spaces is defined by the lower surface of the second solid electrolyte layer 6, the bottom of each of the internal spaces is defined by the upper surface of the first solid electrolyte layer 4, and the lateral surface of each of the internal spaces is defined by the lateral surface of the spacer layer 5.

Each of the first diffusion-rate limiting part 11, the second diffusion-rate limiting part 13, and the third diffusion-rate limiting part 30 is provided as two laterally elongated slits (having the longitudinal direction of the openings in the direction perpendicular to the figure in FIG. 1). Each of the first diffusion-rate limiting part 11, the second diffusion-rate limiting part 13, and the third diffusion-rate limiting part 30 may be in such a form that a desired diffusion resistance is created, but the form is not limited to the slits.

The fourth diffusion-rate limiting part 60 is provided as a single laterally elongated slit (having the longitudinal direction of the opening in the direction perpendicular to the figure in FIG. 1) between the spacer layer 5 and the second solid electrolyte layer 6. The fourth diffusion-rate limiting part 60 may be in such a form that a desired diffusion resistance is created, but the form is not limited to the slits.

Also, at a position farther from the front end than the measurement-object gas flow part 15, a reference gas introduction space 43 is disposed between the upper surface of the third substrate layer 3 and the lower surface of the spacer layer 5 at a position where the reference gas introduction space 43 is laterally defined by the lateral surface of the first solid electrolyte layer 4. The reference gas introduction space 43 has an opening in the other end part (hereinafter, referred to as a rear end part) of the sensor element 101. As a reference gas for NOx concentration measurement, for example, air is introduced into the reference gas introduction space 43.

An air introduction layer 48 is a layer formed of porous alumina, and is so configured that a reference gas is introduced into the air introduction layer 48 via the reference gas introduction space 43. The air introduction layer 48 is formed to cover a reference electrode 42.

The reference electrode 42 is an electrode sandwiched between the upper surface of the third substrate layer 3 and the first solid electrolyte layer 4, and as described above, the air introduction layer 48 leading to the reference gas introduction space 43 is disposed around the reference electrode 42. That is, the reference electrode 42 is disposed to be in contact with a reference gas via the air introduction layer 48 which is a porous material, and the reference gas introduction space 43. As will be described later, the reference electrode 42 can be used to measure the oxygen concentration (oxygen partial pressure) in the first internal cavity 20, the second internal cavity 40, and the third internal cavity 61.

In the measurement-object gas flow part 15, the gas inlet 10 is open to the external space, and the measurement-object gas is taken into the sensor element 101 from the external space through the gas inlet 10.

In the present embodiment, the measurement-object gas flow part 15 is in such a form that the measurement-object gas is introduced through the gas inlet 10 that is open on the front end surface of the sensor element 101, however, the present invention is not limited to this form. For example, the measurement-object gas flow part 15 need not have a recess of the gas inlet 10. In this case, the first diffusion-rate limiting part 11 substantially serves as a gas inlet.

For example, the measurement-object gas flow part 15 may have an opening that communicates with the buffer space 12 or a position in the vicinity of the buffer space 12 of the first internal cavity 20, on a lateral surface along the longitudinal direction of the base part 102. In this case, the measurement-object gas is introduced from the lateral surface along the longitudinal direction of the base part 102 through the opening.

Further, for example, the measurement-object gas flow part 15 may be so configured that the measurement-object gas is introduced through a porous body.

The first diffusion-rate limiting part 11 creates a predetermined diffusion resistance to the measurement-object gas taken through the gas inlet 10.

The buffer space 12 is provided to guide the measurement-object gas introduced from the first diffusion-rate limiting part 11 to the second diffusion-rate limiting part 13.

The second diffusion-rate limiting part 13 creates a predetermined diffusion resistance to the measurement-object gas introduced into the first internal cavity 20 from the buffer space 12.

It suffices that the amount of the measurement-object gas to be introduced into the first internal cavity 20 finally falls within a predetermined range. That is, it suffices that a predetermined diffusion resistance is created in a whole from the front end part of the sensor clement 101 to the second diffusion-rate limiting part 13. For example, the first diffusion-rate limiting part 11 may directly communicate with the first internal cavity 20, or the buffer space 12 and the second diffusion-rate limiting part 13 may be absent.

The buffer space 12 is provided to mitigate the influence of pressure fluctuation on the detected value when the pressure of the measurement-object gas fluctuates.

When the measurement-object gas is introduced from outside the sensor element 101 into the first internal cavity 20, the measurement-object gas, which is rapidly taken through the gas inlet 10 into the sensor element 101 due to pressure fluctuation of the measurement-object gas in the external space (pulsations in exhaust pressure if the measurement-object gas is automotive exhaust gas), is not directly introduced into the first internal cavity 20. Rather, the measurement-object gas is introduced into the first internal cavity 20 after the pressure fluctuation of the measurement-object gas is eliminated through the first diffusion-rate limiting part 11, the buffer space 12, and the second diffusion-rate limiting part 13. Thus, the pressure fluctuation of the measurement-object gas introduced into the first internal cavity 20 becomes almost negligible.

The first internal cavity 20 is provided as a space for adjusting the oxygen partial pressure in the measurement-object gas introduced through the second diffusion-rate limiting part 13. The oxygen partial pressure is adjusted by operation of a main pump cell 21.

The main pump cell 21 is an electrochemical pump cell composed of an inner main pump electrode 22 having a ceiling electrode portion 22a disposed over substantially the entire surface of the lower surface of the second solid electrolyte layer 6 that faces the first internal cavity 20, an outer pump electrode 23 disposed on a region of the upper surface of the second solid electrolyte layer 6 that corresponds to the ceiling electrode portion 22a so as to be exposed to the external space, and the second solid electrolyte layer 6 sandwiched between the inner main pump electrode 22 and the outer pump electrode 23.

The inner main pump electrode 22 is disposed so as to face the first internal cavity 20. That is, the inner main pump electrode 22 is formed to span the upper and lower solid electrolyte layers (the second solid electrolyte layer 6 and the first solid electrolyte layer 4) that define the first internal cavity 20 and the spacer layer 5 that defines the lateral wall. Specifically, the ceiling electrode portion 22a is formed on the lower surface of the second solid electrolyte layer 6 that defines the ceiling surface of the first internal cavity 20, and a bottom electrode portion 22b is formed on the upper surface of the first solid electrolyte layer 4 that defines the bottom surface of the first internal cavity 20. Also, lateral electrode portions (not shown) are formed on the lateral wall surfaces (inner surface) of the spacer layer 5 that form both lateral wall parts of the first internal cavity 20 so as to connect the ceiling electrode portion 22a and the bottom electrode portion 22b. Thus, the inner main pump electrode 22 is provided as a tunnel-like structure in the area where the lateral electrode portions are disposed.

The inner main pump electrode 22 and the outer pump electrode 23 are porous cermet electrodes (electrodes in a state that a metal component and a ceramic component are mixed). The ceramic component to be used is not particularly limited, but is preferably an oxygen-ion-conductive solid electrolyte as in the case of the base part 102. For example, ZrO₂ can be used as the ceramic component.

The inner main pump electrode 22 to be in contact with a measurement-object gas is formed using a material having a weakened reducing ability with respect to a NOx component in the measurement-object gas. The inner main pump electrode 22 preferably contains a noble metal having catalytic activity (e.g., at least one of Pt, Rh, Ir, Ru, and Pd) and a noble metal (e.g., Au, Ag) that reduces the catalytic activity with respect to a target gas to be measured (in this embodiment, NOx) of a noble metal having catalytic activity. In this embodiment, the inner main pump electrode 22 is formed as a porous cermet electrode made of Pt containing 1% of Au and ZrO₂.

The outer pump electrode 23 may contain the above-described noble metal having catalytic activity. Similarly, the reference electrode 42 may contain the above-described noble metal having catalytic activity. In this embodiment, the outer pump electrode 23 is formed as a porous cermet electrode made of Pt and ZrO₂.

In the main pump cell 21, a desired pump voltage Vp0 is applied between the inner main pump electrode 22 and the outer pump electrode 23 by a variable power supply 24 to flow a pump current Ip0 between the inner main pump electrode 22 and the outer pump electrode 23 in either a positive or negative direction, and thus it is possible to pump out oxygen in the first internal cavity 20 to the external space or pump oxygen into the first internal cavity 20 from the external space.

To detect the oxygen concentration (oxygen partial pressure) in the atmosphere in the first internal cavity 20, the inner main pump electrode 22, the second solid electrolyte layer 6, the spacer layer 5, the first solid electrolyte layer 4, the third substrate layer 3, and the reference electrode 42 form an electrochemical sensor cell, namely, an oxygen-partial-pressure detection sensor cell 80 for main pump control.

The oxygen concentration (oxygen partial pressure) in the first internal cavity 20 can be detected from an electromotive force V0 measured in the oxygen-partial-pressure detection sensor cell 80 for main pump control. In addition, the pump current Ip0 is controlled by performing feedback control of the pump voltage Vp0 so that the electromotive force VO is constant. Thus, the oxygen concentration in the first internal cavity 20 can be maintained at a predetermined constant value.

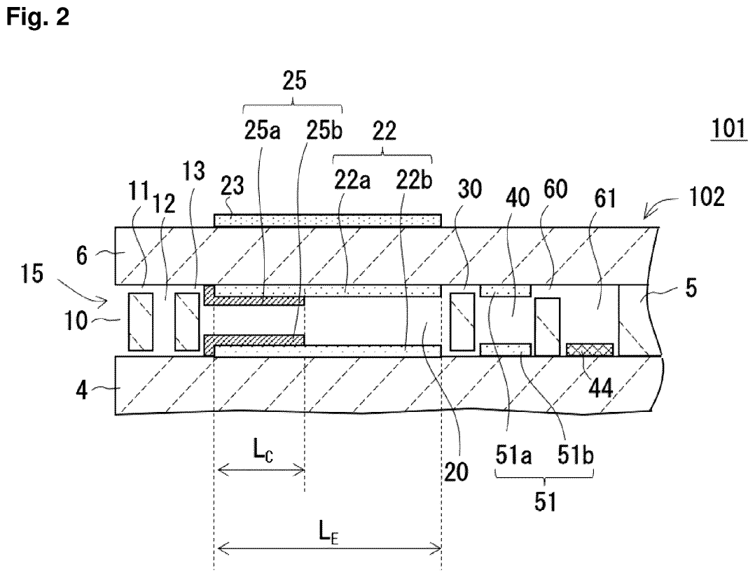

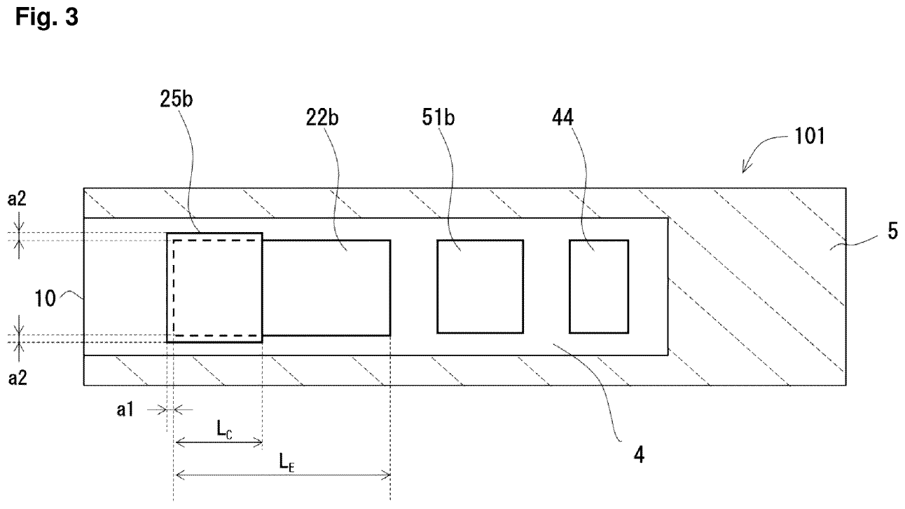

A porous coating layer 25 is disposed so as to cover at least an electrode end part of the inner main pump electrode 22 closer to the one end part (the front end part) in the longitudinal direction of the base part 102. FIG. 2 is a partial sectional schematic view showing an arrangement of the respective electrodes 22, 51, and 44, and the porous coating layer 25 disposed in the measurement-object gas flow part 15. FIG. 3 is a sectional schematic view showing a part of the section along line III-III in FIG. 1. FIG. 3 is a schematic view showing a general planar arrangement of the inner main pump electrode 22, the porous coating layer 25, an auxiliary pump electrode 51, and a measurement electrode 44 disposed on the upper surface of the first solid electrolyte layer 4. A specific configuration of the porous coating layer 25 will be described later.

The third diffusion-rate limiting part 30 creates a predetermined diffusion resistance to the measurement-object gas whose oxygen concentration (oxygen partial pressure) has been controlled in the first internal cavity 20 by the operation of the main pump cell 21 and guides the measurement-object gas into the second internal cavity 40.

The second internal cavity 40 is provided as a space for adjusting the oxygen partial pressure in the measurement-object gas introduced through the third diffusion-rate limiting part 30 more accurately. The oxygen partial pressure is adjusted by operation of an auxiliary pump cell 50.

After the oxygen concentration (oxygen partial pressure) in the measurement-object gas is adjusted in advance in the first internal cavity 20, the measurement-object gas is introduced through the third diffusion-rate limiting part 30, and is further subjected to adjustment of the oxygen partial pressure by the auxiliary pump cell 50 in the second internal cavity 40. Thus, the oxygen concentration in the second internal cavity 40 can be kept constant with high accuracy, and the NOx concentration can be measured with high accuracy in the gas sensor 100.

The auxiliary pump cell 50 is an auxiliary electrochemical pump cell composed of an auxiliary pump electrode 51 having a ceiling electrode portion 51a disposed on substantially the entire surface of the lower surface of the second solid electrolyte layer 6 facing with the second internal cavity 40, the outer pump electrode 23 (the outer electrode is not limited to the outer pump electrode 23, but may be any suitable electrode outside the sensor element 101), and the second solid electrolyte layer 6.

The auxiliary pump electrode 51 is disposed at a position farther from the one end part (the front end part) in the longitudinal direction of the base part 102 (the sensor element 101) than the inner main pump electrode 22 on the inner surface of the measurement-object gas flow part 15.

This auxiliary pump electrode 51 is disposed in the second internal cavity 40 in a tunnel-like structure similar to the inner main pump electrode 22 disposed in the first internal cavity 20. Specifically, in the tunnel-like structure, the ceiling electrode portion 51a is formed on the second solid electrolyte layer 6 that defines the ceiling surface of the second internal cavity 40, a bottom electrode portion 51b is formed on the first solid electrolyte layer 4 that defines the bottom surface of the second internal cavity 40, and lateral electrode portions (not shown) connecting the ceiling electrode portion 51a and the bottom electrode portion 51b are formed on the wall surfaces of the spacer layer 5 that define the lateral walls of the second internal cavity 40.

It is to be noted that the auxiliary pump electrode 51 is formed using a material having a weakened ability to reduce a NOx component in the measurement-object gas, as with the case of the inner main pump electrode 22. The auxiliary pump electrode 51, as with the case of the inner main pump electrode 22, preferably contains a noble metal having catalytic activity (e.g., at least one of Pt, Rh, Ir, Ru, and Pd) and a noble metal (e.g., Au, Ag) that reduces the catalytic activity with respect to a target gas to be measured (in this embodiment, NOx) of a noble metal having catalytic activity. In this embodiment, the auxiliary pump electrode 51 is formed as a porous cermet electrode made of Pt containing 1% of Au and ZrO₂.

In the auxiliary pump cell 50, by applying a desired voltage Vp1 between the auxiliary pump electrode 51 and the outer pump electrode 23 by a variable power supply 52, it is possible to pump out oxygen in the atmosphere in the second internal cavity 40 to the external space, or pump the oxygen into the second internal cavity 40 from the external space.

To control the oxygen partial pressure in the atmosphere in the second internal cavity 40, the auxiliary pump electrode 51, the reference electrode 42, the second solid electrolyte layer 6, the spacer layer 5, the first solid electrolyte layer 4, and the third substrate layer 3 constitute an electrochemical sensor cell, namely, an oxygen-partial-pressure detection sensor cell 81 for auxiliary pump control.

The auxiliary pump cell 50 performs pumping with the variable power supply 52 whose voltage is controlled on the basis of an electromotive force VI detected by the oxygen-partial-pressure detection sensor cell 81 for auxiliary pump control. Thus, the oxygen partial pressure in the atmosphere in the second internal cavity 40 is controlled to such a low partial pressure that does not substantially affect measurement of NOx.

In addition, a pump current Ip1 in the auxiliary pump cell 50 is used for control of the electromotive force V0 of the oxygen-partial-pressure detection sensor cell 80 for main pump control. Specifically, the pump current Ip1 is input to the oxygen-partial-pressure detection sensor cell 80 for main pump control as a control signal to control the electromotive force V0, and thus the gradient of the oxygen partial pressure in the measurement-object gas introduced into the second internal cavity 40 from the third diffusion-rate limiting part 30 is controlled to remain constant. In using as a NOx sensor, the oxygen concentration in the second internal cavity 40 is kept at a constant value of about 0.001 ppm by the actions of the main pump cell 21 and the auxiliary pump cell 50.

The fourth diffusion-rate limiting part 60 creates a predetermined diffusion resistance to the measurement-object gas whose oxygen concentration (oxygen partial pressure) has been controlled to further low in the second internal cavity 40 by the operation of the auxiliary pump cell 50, and guides the measurement-object gas into the third internal cavity 61.

The third internal cavity 61 is provided as a space for measuring nitrogen oxide (NOx) concentration in the measurement-object gas introduced through the fourth diffusion-rate limiting part 60. By the operation of a measurement pump cell 41, NOx concentration is measured.

The measurement pump cell 41 measures NOx concentration in the measurement-object gas in the third internal cavity 61. The measurement pump cell 41 is an electrochemical pump cell composed of a measurement electrode 44 disposed on the upper surface of the first solid electrolyte layer 4 facing with the third internal cavity 61, the outer pump electrode 23 (the outer electrode is not limited to the outer pump electrode 23, but may be any suitable electrode outside the sensor element 101), the second solid electrolyte layer 6, the spacer layer 5, and the first solid electrolyte layer 4.

The measurement electrode 44 is disposed at a position farther from the one end part (the front end part) in the longitudinal direction of the base part 102 (the sensor element 101) than the inner main pump electrode 22 and the auxiliary pump electrode 51 on the inner surface of the measurement-object gas flow part 15.

The measurement electrode 44 is a porous cermet electrode. The measurement electrode 44 functions also as a NOx reduction catalyst that reduces NOx present in the atmosphere in the third internal cavity 61. The measurement electrode 44 is an electrode containing a noble metal having catalytic activity (e.g., at least one of Pt, Rh, Ir, Ru, and Pd). It is preferred that the measurement electrode 44 does not contain a noble metal (e.g., Au, Ag) that reduces the catalytic activity with respect to a target gas to be measured (in this embodiment, NOx) of a noble metal having catalytic activity. In this embodiment, the measurement electrode 44 is formed as a porous cermet electrode made of Pt and Rh, and ZrO₂.

In the measurement pump cell 41, oxygen generated by decomposition of nitrogen oxide in the atmosphere around the measurement electrode 44 is pumped out, and the amount of generated oxygen can be detected as a pump current Ip2.

To detect the oxygen partial pressure around the measurement electrode 44, the first solid electrolyte layer 4, the third substrate layer 3, the measurement electrode 44, and the reference electrode 42 constitute an electrochemical sensor cell, namely an oxygen-partial-pressure detection sensor cell 82 for measurement pump control. A variable power supply 46 is controlled on the basis of an electromotive force V2 detected by the oxygen-partial-pressure detection sensor cell 82 for measurement pump control.

The measurement-object gas introduced into the second internal cavity 40 reaches the measurement electrode 44 in the third internal cavity 61 through the fourth diffusion-rate limiting part 60 under the condition that the oxygen partial pressure is controlled. Nitrogen oxide in the measurement-object gas around the measurement electrode 44 is reduced (2NO→N₂+O₂) to generate oxygen. The generated oxygen is to be pumped by the measurement pump cell 41, and at this time, a voltage Vp2 of the variable power supply 46 is controlled so that the electromotive force V2 detected by the oxygen-partial-pressure detection sensor cell 82 for measurement pump control is constant. Since the amount of oxygen generated around the measurement electrode 44 is proportional to the concentration of nitrogen oxide in the measurement-object gas, nitrogen oxide concentration in the measurement-object gas is calculated by using the pump current Ip2 in the measurement pump cell 41.

By configuring oxygen partial pressure detecting means by an electrochemical sensor cell composed of a combination of the measurement electrode 44, the first solid electrolyte layer 4, the third substrate layer 3 and the reference electrode 42, it is possible to detect an electromotive force in accordance with a difference between the amount of oxygen generated by reduction of NOx components in the atmosphere around the measurement electrode 44 and the amount of oxygen contained in the reference air, and hence it is possible to determine the concentration of NOx components in the measurement-object gas.

Also, the second solid electrolyte layer 6, the spacer layer 5, the first solid electrolyte layer 4, the third substrate layer 3, the outer pump electrode 23, and the reference electrode 42 constitute an electrochemical sensor cell 83, and it is possible to detect the oxygen partial pressure in the measurement-object gas outside the sensor by an electromotive force Vref obtained by the sensor cell 83.

In the gas sensor 100 having such a configuration, the main pump cell 21 and the auxiliary pump cell 50 are operated to supply a measurement-object gas whose oxygen partial pressure is usually kept at a low constant value (the value that does not substantially affect measurement of NOx) to the measurement pump cell 41. Therefore, NOx concentration in the measurement-object gas can be detected on the basis of the pump current Ip2 that flows as a result of pumping out of the oxygen generated by reduction of NOx by the measurement pump cell 41 and is almost in proportion to the concentration of NOx in the measurement-object gas.

The sensor element 101 further includes a heater part 70 that functions as a temperature regulator of heating and maintaining the temperature of the sensor element 101 so as to enhance the oxygen ion conductivity of the solid electrolyte. The heater part 70 includes a heater electrode 71, a heater 72, a heater lead 76, a through hole 73, a heater insulating layer 74, and a pressure relief vent 75.

The heater electrode 71 is an electrode formed in contact with the lower surface of the first substrate layer 1. The power can be supplied to the heater part 70 from the outside by connecting the heater electrode 71 with a heater power supply that is an external power supply.

The heater 72 is an electrical resistor sandwiched by the second substrate layer 2 and the third substrate layer 3 from top and bottom. The heater 72 is connected with the heater electrode 71 via the heater lead 76 that connects with the heater 72 and extends in the rear end side in the longitudinal direction of the sensor element 101, and the through hole 73. The heater 72 is externally powered through the heater electrode 71 to generate heat, and heats and maintains the temperature of the solid electrolyte forming the sensor element 101.

The heater 72 is embedded over the whole area from the first internal cavity 20 to the third internal cavity 61 so that the temperature of the sensor element 101 can be adjusted to such a temperature that activates the solid electrolyte. The temperature may be adjusted so that the main pump cell 21, the auxiliary pump cell 50, and the measurement pump cell 41 are operable. It is not necessary that the whole area is adjusted to the same temperature, but the sensor element 101 may have temperature distribution.

In the sensor element 101 of the present embodiment, the heater 72 is embedded in the base part 102, but this form is not limitative. The heater 72 may be disposed to heat the base part 102. That is, the heater 72 may heat the sensor element 101 to develop oxygen ion conductivity with which the main pump cell 21, the auxiliary pump cell 50, and the measurement pump cell 41 are operable. For example, the heater 72 may be embedded in the base part 102 as in the present embodiment. Alternatively, for example, the heater part 70 may be formed as a heater substrate that is separate from the base part 102, and may be disposed at a position adjacent to the base part 102.

The heater insulating layer 74 is formed of an insulator such as alumina on the upper and lower surfaces of the heater 72 and the heater lead 76. The heater insulating layer 74 is formed to ensure electrical insulation between the second substrate layer 2, and the heater 72 and the heater lead 76, and electrical insulation between the third substrate layer 3, and the heater 72 and the heater lead 76.

The pressure relief vent 75 extends through the third substrate layer 3 so that the heater insulating layer 74 and the reference gas introduction space 43 communicate with each other. The pressure relief vent 75 can mitigate an increase in internal pressure due to temperature rise in the heater insulating layer 74. The pressure relief vent 75 may be absent.

### Current Flowing Through Main Pump Cell

As described above, the main pump cell 21 discharges oxygen from the first internal cavity 20 so that the oxygen concentration in the first internal cavity 20 is at a predetermined constant value. The higher the oxygen concentration in the measurement-object gas, the larger the amount of oxygen to be discharged by the main pump cell 21. That is, the pump current Ip0 flowing through the main pump cell 21 increases. The larger the pump current Ip0 flowing through the main pump cell 21, the higher the pump voltage Vp0 in the main pump cell 21. That is, the higher the oxygen concentration in the measurement-object gas, the more the pump voltage Vp0 increases.

If the pump voltage Vp0 is too high, NOx may be decomposed in the inner main pump electrode 22. This leads to reduction in the amount of NOx reaching the measurement electrode 44. As a result, a value of the pump current Ip2 detected by the measurement pump cell 41 is smaller than the value that is to be originally detected. Especially when oxygen concentration of the measurement-object gas is high, the detection accuracy of NOx may decrease.

When NOx is decomposed in the inner main pump electrode 22, the detection accuracy of NOx may decrease under high oxygen concentration. Degree of NOx decomposition in the inner main pump electrode 22 can be evaluated based on degree of linearity between oxygen concentration and the pump current Ip2 (namely, a NOx output current). The degree of the linearity between the oxygen concentration and the pump current Ip2 (the NOx output current) can be evaluated by using a coefficient of determination R² (i.e., a square value of a correlation coefficient) in a linear regression equation between plural oxygen concentrations and the respective Ip2 values at the respective oxygen concentrations. The coefficient of determination R² is also written as linearity R² of NOx output with respect to oxygen concentration.

The higher the linearity R² of the NOx output with respect to the oxygen concentration, namely, the closer to 1 the linearity R² is, with the higher accuracy NOx can be detected regardless of the oxygen concentration in the measurement-object gas. The linearity R² of the NOx output with respect to the oxygen concentration may be, for example, 0.900 or more. It is expected that NOx can be measured with high accuracy in actual use by using such a gas sensor. More preferably, the linearity R² of the NOx output with respect to the oxygen concentration may be 0.950 or more. Further preferably, the linearity R² of the NOx output with respect to the oxygen concentration may be 0.960 or more, or, 0.975 or more.

The linearity R² (the coefficient of determination R²) of the NOx output with respect to the oxygen concentration can be calculated, for example, by using a model gas. Four kinds of model gas each having a constant NOx concentration of 500 ppm and an oxygen concentration of 0, 5, 10, or 18% may be subjected to measurement by the gas sensor 100. A coefficient of determination R² may be calculated in a linear regression equation between respective oxygen concentrations of the model gas, and the measured four NOx output current values Ip2. The model gas is not limited to these four kinds, but may be appropriately selected depending on the use modes that are assumed for the gas sensor 100.

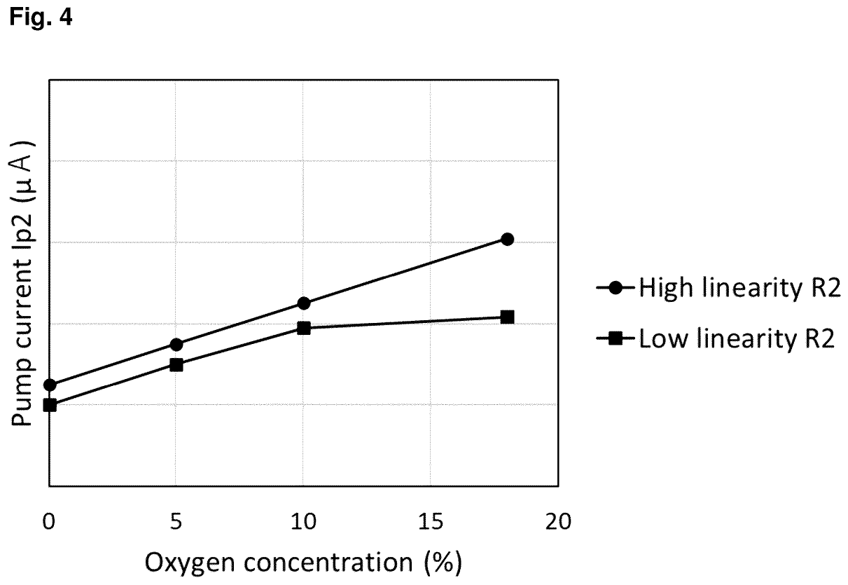

FIG. 4 is a schematic diagram showing the relation between the oxygen concentration and the pump current Ip2 in the presence of oxygen (O₂=0, 5, 10, 18%). In FIG. 4, the black circle “●” schematically indicates a pump current Ip2 in a gas sensor capable of measuring with high accuracy even at high oxygen concentration, namely, a gas sensor having high linearity R² of the NOx output with respect to the oxygen concentration. The black square “▪” schematically shows a pump current Ip2 in a gas sensor having low detection accuracy of NOx at high oxygen concentration, namely, a gas sensor having low linearity R² of the NOx output with respect to the oxygen concentration.

In the gas sensor having high linearity R² of the NOx output with respect to the oxygen concentration, as shown by the black circle “●”, a linear relationship is observed where the pump current Ip2 (the NOx output current) increases monotonically with respect to oxygen concentration. On the other hand, if NOx is decomposed at the inner main pump electrode 22 and an amount of NOx reaching the measurement electrode 44 decreases as described above when the oxygen concentration in the measurement-object gas is high, the pump current Ip2 does not increase at high oxygen concentration and the linearity R² of the NOx output with respect to the oxygen concentration becomes low, as shown by the black square “▪”.

As shown by the black circle “●”, it is considered that the value of the pump current Ip2 (the NOx output current) tends to depend on the oxygen concentration in the measurement-object gas. This suggests that, in determining the NOx concentration from the pump current Ip2, correction using the oxygen concentration is effective to determine the NOx concentration with more accuracy. Such correction can be achieved, for example, by correcting the pump current Ip2 based on information (e.g., the pump current Ip0 and the electromotive force Vref) indicating the oxygen concentration in the measurement-object gas.

The linearity R² of the NOx output is supposed to decrease as the gas sensor 100 is used for a long term. By suppressing the decrease in the linearity R² of the NOx output due to use of the gas sensor, it is believed that the decrease in NOx detection accuracy due to long-term use of the gas sensor can be suppressed.

The inner main pump electrode 22 is disposed from a closer side to the one end part (the front end part) toward a farther side from the one end part (the front end part) in the longitudinal direction of the base part 102 (the sensor element 101) on the inner surface of the measurement-object gas flow part 15.

When the measurement-object gas reaches the inner main pump electrode 22, oxygen O₂ in the measurement-object gas enters into pores of the porous inner main pump electrode 22. When the oxygen O₂ touches, in the pores and on the surface of the inner main pump electrode 22, the catalytic metal (Pt in this embodiment) that constitutes the inner main pump electrode 22, the oxygen O₂ is converted to oxygen ion O²⁻. This oxygen ion passes through the solid electrolyte layer (e.g., the second solid electrolyte layer 6) and is released to the outside. In this way, the oxygen O₂ is pumped from the first internal cavity 20 over the whole of the inner main pump electrode 22.

The measurement-object gas introduced into the measurement-object gas flow part 15 from the gas inlet 10 first reaches, in the first internal cavity 20, one electrode end part (namely, an electrode front end part) of the inner main pump electrode 22 closer to the front end part of the base part 102 (the sensor element 101). And, the measurement-object gas flows toward the other electrode end part (namely, an electrode rear end part) of the inner main pump electrode 22 farther from the front end part of the base part 102, while contacting the inner main pump electrode 22. Oxygen O₂ in the measurement-object gas in contact with the inner main pump electrode 22 is sequentially pumped out as the pump current Ip0 flowing through the main pump cell 21. As a result, oxygen concentration in the measurement-object gas decreases from the electrode front end part toward the electrode rear end part of the inner main pump electrode 22. In other words, oxygen concentration is high in the measurement-object gas in contact with the electrode front end part of the inner main pump electrode 22, and oxygen concentration is low in the measurement-object gas in contact with the electrode rear end part of the inner main pump electrode 22. Therefore, microscopically, it is considered that more oxygen is pumped out at the electrode front end part of the inner main pump electrode 22, thereby causing current concentration. Current density is not uniform across the entire inner main pump electrode 22, and it is considered that the current density is usually maximum at the electrode front end part of the inner main pump electrode 22, and decreases toward the electrode rear end part of the inner main pump electrode 22.

When the current concentration as described above occurs, it is considered that since a large pump current Ip0 flows locally at the electrode front end part of the inner main pump electrode 22, Pt and Au contained in the inner main pump electrode 22 are likely to evaporate locally at the electrode front end part. Pt is an example of the noble metal having catalytic activity, and Au is an example of the noble metal that reduces the catalytic activity with respect to NOx of the noble metal having catalytic activity. When Pt and Au evaporates, reaction resistance locally increases at a portion of the inner main pump electrode 22 (mainly, at the electrode front end part) where Pt and Au has decreased due to evaporation. This causes increase in resistance of the inner main pump electrode 22 as a whole. Thus, the pump voltage Vp0 applied to the entire inner main pump electrode 22 increases. As a result, it may occur that at the portion of the inner main pump electrode 22 (mainly, at the electrode front end part) where Pt and Au has decreased due to evaporation, NOx in the measurement-object gas may be easily decomposed due to decrease in Au. In addition, as the pump voltage Vp0 increases, the current concentration is considered to further increase at the portion of the inner main pump electrode 22 (mainly, at the electrode front end part) where Pt and Au has decreased due to evaporation. In particular, when the gas sensor is used for a long period of time, the local evaporation of Pt and Au due to the current concentration, the resulting increase in the pump voltage Vp0, and further current concentration as described above are assumed to occur continuously. Therefore, the pump voltage Vp0 is considered to become larger with use of the gas sensor 100. As a result, NOx is considered to be easily decomposed at the electrode front end part of the inner main pump electrode 22. That is, the linearity R² of the NOx output with respect to the oxygen concentration is considered to decrease with use of the gas sensor 100.

By mitigating the current concentration at the electrode front end part of the inner main pump electrode 22, it is considered that a maximum current density in the inner main pump electrode 22 can be lowered even if an average current density in the inner main pump electrode 22 is the same. When the maximum current density is small, local evaporation of Pt and Au can be suppressed so that the pump voltage Vp0 can be prevented from becoming too large. This is thought to result in suppressing NOx decomposition at the inner main pump electrode 22. The present inventors found that the current concentration can be mitigated by making it difficult for oxygen O₂ in measurement-object gas to come in contact with the catalytic metal in the inner main pump electrode 22.

### (Porous Coating Layer)

The porous coating layer 25 is a porous body formed for mitigating the above-described current concentration. As described above, the porous coating layer 25 is disposed so as to cover at least an electrode end part of the inner main pump electrode 22 closer to the one end part (the front end part) in the longitudinal direction of the base part 102.

As described above, FIG. 2 is the partial sectional schematic view showing the arrangement of the respective electrodes 22, 51, and 44, and the porous coating layer 25 disposed in the measurement-object gas flow part 15. Also, as described above, FIG. 3 is the sectional schematic view showing the part of the section along line III-III in FIG. 1. FIG. 3 is the schematic view showing the general planar arrangement of the inner main pump electrode 22 (22b), the porous coating layer 25 (25b), the auxiliary pump electrode 51 (51b), and the measurement electrode 44 disposed on the upper surface of the first solid electrolyte layer 4. From each electrode toward the rear end of the element, an electrode lead which is not shown is disposed to allow connection with the outside. In FIG. 3, the spacer layer 5 forming the first diffusion-rate limiting part 11, the second diffusion-rate limiting part 13, the third diffusion-rate limiting part 30, and the fourth diffusion-rate limiting part 60 is omitted in the drawing. In FIG. 2 and FIG. 3, L_{E} indicates a length of the inner main pump electrode 22 in the longitudinal direction of the sensor element 101, and L_{C} indicates a length of a region (a region illustrated by a dashed line in FIG. 3) of the inner main pump electrode 22 covered by the porous coating layer 25 in the longitudinal direction of the sensor element 101.

In this embodiment, the porous coating layer 25 is composed of:
a ceiling coating layer 25a covering a region of the ceiling electrode portion 22a in the inner main pump electrode 22 including an electrode end part (an electrode front end part) closer to the front end part of the sensor element 101, and having a length Le in the longitudinal direction of the sensor element 101, and
a bottom coating layer 25b covering a region of the bottom electrode portion 22b in the inner main pump electrode 22 including an electrode end part (an electrode front end part) closer to the front end part of the sensor element 101, and having a length Le in the longitudinal direction of the sensor element 101.

When the inner main pump electrode 22 has the ceiling electrode portion 22a and the bottom electrode portion 22b, the porous coating layer 25 may be formed to cover the electrode front end part in at least one of the electrode portions. When the porous coating layer 25 covers at least the electrode front end part of either the ceiling electrode portion 22a or the bottom electrode portion 22b in the inner main pump electrode 22, the above-described effect of mitigating the current concentration is expected. The porous coating layer 25 may cover at least the electrode front end part of the electrode portion (for example, the ceiling electrode portion 22a) at which the current concentration is greater. Alternatively, the ceiling coating layer 25a and the bottom coating layer 25b in the porous coating layer 25 may be formed to cover the respective electrode front end parts of both of the ceiling electrode portion 22a and the bottom electrode portion 22b in the inner main pump electrode 22. In this case, the above-described effect of mitigating the current concentration can be more expected.

In this embodiment, both of the ceiling coating layer 25a and the bottom coating layer 25b in the porous coating layer 25 is configured to cover the region of the length L_{C} in the longitudinal direction of the sensor element 101 from the electrode front end part. However, the porous coating layer 25 is not limited thereto. The ceiling coating layer 25a and the bottom coating layer 25b may have a different length from each other.

In this embodiment, as shown in FIG. 3, the inner main pump electrode 22 is an electrode having a substantially rectangular shape in planar view. The porous coating layer 25 covers a region of the length L_{C} in the longitudinal direction of the sensor element 101 from an electrode end of the inner main pump electrode 22 close to the front end part of the sensor element 101, in the planar view.

The porous coating layer 25 may be formed to cover at least the electrode front end part of the inner main pump electrode 22. By covering the electrode front end part, an amount of oxygen O₂ reaching the electrode front end part of the inner main pump electrode 22 can be decreased, thereby reducing the current concentration on the electrode front end part. The porous coating layer 25 may cover a region in the inner main pump electrode 22 including the electrode front end part, and having a predetermined length in the longitudinal direction of the base part 102.

For example, the porous coating layer 25 may be formed to cover an area including the electrode front end part of the inner main pump electrode 22, and having 3% or more of area of the inner main pump electrode 22 (when the inner main pump electrode 22 is formed on both top and bottom surfaces, each of the ceiling electrode portion 22a and/or the bottom electrode portion 22b). That is, an area ratio of the region of the inner main pump electrode 22 covered by the porous coating layer 25 to the inner main pump electrode 22 may be 3% or more. Alternatively, the area ratio may be 5% or more, 10% or more, 20% or more, or the like. The porous coating layer 25 may entirely cover the inner main pump electrode 22. That is, the area ratio of the region of the inner main pump electrode 22 covered by the porous coating layer 25 to the inner main pump electrode 22 may be 100% or less. Alternatively, the area ratio may be 90% or less, or, 75% or less. It is to be noted that area of the part of the inner main pump electrode 22 covered by the porous coating layer 25 is roughly equal to area of the porous coating layer 25.

Here, the area of the inner main pump electrode 22 means the area of the inner main pump electrode 22 in planar view, that is, the area of the inner main pump electrode 22 in FIG. 3. When the inner main pump electrode 22 and the porous coating layer 25 are substantially rectangular in shape, an area ratio of area of the part of the inner main pump electrode 22 covered by the porous coating layer 25 to area of the inner main pump electrode 22 is approximately the same as a ratio of the length L_{C} in the longitudinal direction of the part of the inner main pump electrode 22 covered by the porous coated layer 25 to the length L_{E} in the longitudinal direction of the inner main pump electrode 22. That is, the porous coating layer 25 may be formed to cover a region in the inner main pump electrode 22 including the electrode front end part, and having the length L_{C} that is 3% or more of the length L_{E} of the inner main pump electrode 22.

The porous coating layer 25 may cover an end surface on the front end side of the inner main pump electrode 22 as shown in FIG. 2 and FIG. 3, or may not cover the end surface. In FIG. 2, a cross section of the inner main pump electrode 22 is illustrated as a rectangle, but is not limited to this. The corners of end parts of the inner main pump electrode 22 may not have a right angle and may be rounded. The inner main pump electrode 22 may have a gently sloping shape with no clear edge, or the like. Regardless of the cross-sectional shape of the inner main pump electrode 22, the electrode end part of the inner main pump electrode 22 closer to the front end part of the sensor element 101 may be covered by the porous coating layer 25, or may be exposed. For example, as shown in FIG. 3, the porous coating layer 25 may have a shape that is longer by a length al toward the front end side of the sensor element 101 from the inner main pump electrode 22. The length al may be appropriately determined, and may be, for example, roughly the same as a thickness of the porous coating layer 25.

Further, the porous coating layer 25 may cover both end surfaces in a direction perpendicular to the longitudinal direction (in a width direction of the sensor element 101) of the inner main pump electrode 22, or may not cover the both end surfaces. For example, as shown in FIG. 3, the porous coating layer 25 may have a shape that is longer by a length a2 toward each of left and right sides in the width direction of the sensor element 101 from the inner main pump electrode 22. The length a2 may be appropriately determined, and may be, for example, roughly the same as a thickness of the porous coating layer 25. The length a2 may be the same on the left and right sides, or may be different between the left and right sides.

The porous coating layer 25 is a porous body. A constituent material of the porous coating layer 25 may be a material that does not substantially include a catalyst metal. Examples of the constituent material of the porous coating layer 25 include alumina, zirconia, spinel, cordierite, mullite, titania, and magnesia. Any one or two or more of them may be used. In this embodiment, the porous coating layer 25 comprises an alumina porous material.

Preferably, an oxygen diffusion coefficient of the porous coating layer 25 may be 1×10⁻⁶ m²/s or less in at least a part of the porous coating layer 25. More preferably, the oxygen diffusion coefficient may be 1×10⁻⁶ m²/s in a part of the porous coating layer 25 covering a surface of the electrode front end part of the inner main pump electrode 22. When the oxygen diffusion coefficient is within such a range, it is considered that the porous coating layer 25 can make oxygen in the measurement-object gas harder to reach the inner main pump electrode 22, thereby further mitigating the current concentration on the electrode front end part in the inner main pump electrode 22.

The oxygen diffusion coefficient of the porous coating layer 25 may be 2×10⁻⁹ m²/s or more. If the diffusion coefficient is extremely too small, oxygen hardly reaches the part of the inner main pump electrode 22 covered by the porous coating layer 25, which may cause the current concentration on the front end side of a part of the inner main pump electrode 22 not covered by the porous coating layer 25.

The diffusion coefficient of the porous coating layer 25 can be determined in the following manner. For example, measurement of diffusion resistance is performed by using a sensor element for measurement in which the porous coating layer 25 is formed on an entire surface of the measurement electrode 44 in the sensor element 101. Specifically, the sensor clement for measurement is heated to a temperature at which the solid electrolyte is activated, and a current-voltage curve is measured between the measurement electrode 44 and the outer pump electrode 23. A limiting current value is obtained from the current-voltage curve to calculate diffusion resistance. Effects of diffusion resistance of the diffusion-rate limiting parts 11, 13, 30, and 60 and the internal cavities 20, 40, and 61 is excluded from the calculated diffusion resistance to obtain diffusion resistance of the porous coating layer 25. A diffusion coefficient of a porous body forming the porous coating layer 25 is calculated from the obtained diffusion resistance of the porous coating layer 25. The diffusion coefficient can be obtained in the same manner by using a sensor element for measurement in which the porous coating layer 25 is formed on an entire surface of the inner main pump electrode 22 or the auxiliary pump electrode 51.

The oxygen diffusion coefficient of the porous coating layer 25 is roughly correlated with a porosity of the porous coating layer 25. When alumina is used as the constituent material of the porous coating layer 25, 1×10⁻⁶ m²/s of the oxygen diffusion coefficient corresponds to roughly 20% as the porosity of the porous coating layer 25. 7×10⁻⁸ m²/s of the oxygen diffusion coefficient corresponds to roughly 10% as the porosity of the porous coating layer 25. A value of the porosity may vary depending on the constituent material of the porous coating layer 25, but may be appropriately determined by a person skilled in the art. When alumina is used as the constituent material of the porous coating layer 25, the porosity of the porous coating layer 25 may be, for example, about 20% or less. The porosity may be, for example, about 3% or more. The oxygen diffusion coefficient of the porous coating layer 25 may differ depending on constituent materials of the porous coating layer 25. The oxygen diffusion coefficient of the porous coating layer 25 may be appropriately varied by changing the constituent material and/or the porosity of the porous coating layer 25 properly.

The porosity of the porous coating layer 25 is determined in the following manner using an image (SEM image) obtained by observation with a scanning electron microscope (SEM). In an area where the porous coating layer 25 is present, the sensor element 101 is cut in the longitudinal direction of the sensor element 101. The cut surface is embedded in a resin and polished to prepare an observation sample. The magnification of the SEM is set to 80 times, and the surface to be observed of the observation sample is imaged to obtain an SEM image of section of the porous coating layer 25. Then, the obtained SEM image is binarized using “Otsu's method” (also referred to as discriminant analysis method). In the binarized image, alumina is shown in white and pores are shown in black. In the binarized image, area of alumina portions (white) and area of pore portions (black) are obtained. The ratio of the area of the pore portions to total area (total of the area of the alumina portions and the area of the pore portions) is calculated and defined as porosity. In this embodiment, the porous coating layer 25 is considered to have substantially the same microstructure regardless of observation area. Therefore, as described above, the porosity determined using one sectional image may be used as the porosity of the porous coating layer 25.

The oxygen diffusion coefficient of the porous coating layer 25 may be the same over the entire porous coating layer 25, or, may differ (vary) in the longitudinal direction of the base part 102. The oxygen diffusion coefficient of the porous coating layer 25 may differ (vary) in the width direction perpendicular to the longitudinal direction of the base part 102. Alternatively, the porous coating layer 25 may be composed of multiple layers that have different diffusion coefficients for oxygen O₂ respectively.

The porous coating layer 25 may be so configured that the oxygen diffusion coefficient of the porous coating layer 25 is stepwise or continuously increased from a closer side toward a farther side with respect of the one end part (the front end part) in the longitudinal direction of the base part 102. As described above, oxygen concentration in the measurement-object gas decreases from the electrode front end part toward the electrode rear end part of the inner main pump electrode 22. Therefore, by making the diffusion coefficient for oxygen O₂ increase stepwise or continuously from the electrode front end part toward the electrode rear end part of the inner main pump electrode 22, it is considered that current density distribution in the inner main pump electrode 22 approaches a more uniform state, and the current concentration on the electrode front end part in the inner main pump electrode 22 can be mitigated more effectively.

Preferably, a thickness of the porous coating layer 25 may be 1 μm or more. As the thickness gets thicker, an amount of oxygen reaching the inner main pump electrode 22 is limited on the part of the inner main pump electrode 22 covered by the porous coating layer 25. When the thickness of the porous coating layer 25 is 1 μm or more, the effect of mitigating the current concentration on the electrode front end part in the inner main pump electrode 22 is further obtained.

An upper limit of the thickness of the porous coating layer 25 may be a thickness with which gas diffusion in the longitudinal direction of the sensor element 101 is not inhibited in the first internal cavity 20. The thickness of the porous coating layer 25 may vary depending on the configuration of the measurement-object gas flow part 15, but may be, for example, 45 μm or less.

The thickness of the porous coating layer 25 is determined in the following manner using an image (SEM image) obtained by observation with a scanning electron microscope (SEM). As in the case of determination of the porosity described above, the magnification of the SEM is set to 80 times, and the SEM image of section of the porous coating layer 25 is obtained. A direction perpendicular to the longitudinal direction of the sensor element 101 is defined as a thickness direction, a distance between the surface of the porous coating layer 25 and the interface with the inner main pump electrode 22 is determined, and the distance is defined as the thickness of the porous coating layer 25. It is to be noted that, when the porous coating layer 25 is formed as a uniform layer having a predetermined thickness, the thickness determined using one sectional image may be used as the thickness of the porous coating layer 25.

An amount of oxygen that reaches the inner main pump electrode 22 from a surface of the porous coating layer 25 is considered to vary by the diffusion coefficient and the thickness of the porous coating layer 25. More preferable range of the thickness may be appropriately determined depending on a value of the oxygen diffusion coefficient. Also, more preferable range of the oxygen diffusion coefficient may be appropriately determined depending on the thickness. These may allow an amount of oxygen that reaches the inner main pump electrode 22 from a surface of the porous coating layer 25 to be a more preferable amount.

Further, the porous coating layer 25 has a function of suppressing evaporation of Au from the region of the inner main pump electrode 22 covered by the porous coating layer 25. When the gas sensor is used for a long term under high oxygen concentration in a high temperature range, it is assumed that the Au in the inner main pump electrode 22 evaporates and adheres to the measurement electrode 44. As a result, NOx decomposition activity in the measurement electrode 44 deteriorates and detection accuracy of the NOx sensor is decreased. Au is considered to evaporate more likely at high oxygen concentration and a high temperature. At the electrode end part (the electrode front end part) of the inner main pump electrode 22 closer to the front end part of the sensor element 101, Au is considered to be likely to evaporate since oxygen concentration in the measurement-object gas in the vicinity of the electrode front end part is high. The porous coating layer 25 covers at least the electrode front end part of the inner main pump electrode 22. It is considered that the evaporation of Au from the inner main pump electrode 22 can be effectively suppressed by covering the electrode front end part of the inner main pump electrode 22, where the evaporation of Au is likely to occur, with the porous coating layer 25. As a result, it is considered that the deterioration of NOx decomposition activity in the measurement electrode 44 can be effectively suppressed.

Further, when Au evaporates from the inner main pump electrode 22, an amount of Au contained in the inner main pump electrode 22 decreases. The decrease in the amount of Au is considered to deteriorate the effect by Au of suppressing the catalytic activity to decompose NOx. As a result, it is concerned that NOx decomposition at the inner main pump electrode 22 is promoted. As described above, it is considered that the evaporation of Au from the inner main pump electrode 22 can be effectively suppressed by covering the electrode front end part of the inner main pump electrode 22, where the evaporation of Au is likely to occur, with the porous coating layer 25. As a result, it is considered that NOx decomposition at the inner main pump electrode 22 is more suppressed.

The sensor element 101 for detecting NOx concentration in a measurement-object gas have been described above as an example of the embodiment according to the present invention, but the present invention is not limited thereto. The present invention may include a sensor clement having any structure as long as the object of the present invention can be achieved, that is, decrease in detection accuracy due to long-term use of a gas sensor is suppressed.

In the above embodiment, the gas sensor 100 detects the NOx concentration in a measurement-object gas. However, the target gas to be measured is not limited to NOx. For example, the target gas to be measured may be an oxide gas other than NOx (e.g., carbon dioxide CO₂, water H₂O). When the target gas to be measured is an oxide gas, as in the case of the above embodiment in which the NOx concentration is detected, a measurement-object gas containing an oxide gas itself is introduced into the third internal cavity 61, and the oxide gas in the measurement-object gas is reduced at the measurement electrode 44 so that oxygen is generated. The target gas to be measured can be detected by acquiring the generated oxygen as the pump current Ip2 in the measurement pump cell 41.

Alternatively, for example, the target gas to be measured may be a non-oxide gas such as ammonia NH₃. When the target gas to be measured is a non-oxide gas, the non-oxide gas is converted to an oxide gas (for example, in the case of ammonia NH₃, NH₃ is converted to NO), and a measurement-object gas containing the converted oxide gas is introduced into the third internal cavity 61. At the measurement electrode 44, the converted oxide gas in the measurement-object gas is reduced so that oxygen is generated. The target gas to be measured can be detected by acquiring the generated oxygen as the pump current Ip2 in the measurement pump cell 41. The conversion from the non-oxide gas to the oxide gas can be performed by allowing at least one of the inner main pump electrode 22 and the auxiliary pump electrode 51 to function as a catalyst.

In the gas sensor 100 of the above embodiment, the inner main pump electrode 22 is composed of the ceiling electrode portion 22a formed on the ceiling surface of the first internal cavity 20, the bottom electrode portion 22b formed on the bottom surface of the first internal cavity 20, and the lateral electrode portions formed on the lateral wall surfaces of the first internal cavity 20 so as to connect the ceiling electrode portion 22a and the bottom electrode portion 22b. However, the inner main pump electrode 22 is not limited thereto. For example, the inner main pump electrode 22 may be formed only on the ceiling surface of the first internal cavity 20. Alternatively, the inner main pump electrode 22 may be formed only on the bottom surface of the first internal cavity 20. For example, when the inner main pump electrode 22 has the ceiling electrode portion 22a and the bottom electrode portion 22b, the ceiling electrode portion 22a and the bottom electrode portion 22b may be the same in size, or the size of the ceiling electrode portion 22a and the bottom electrode portion 22b are different from each other. The same is true for the auxiliary pump electrode 51.

In the gas sensor 100 of the above embodiment, as shown in FIG. 1, the sensor element 101 has a structure in which three internal cavities, the first internal cavity 20, the second internal cavity 40, and the third internal cavity 61 are provided and the inner main pump electrode 22, the auxiliary pump electrode 51, and the measurement electrode 44 are respectively disposed in these internal cavities. However, the structure of the sensor element 101 is not limited thereto. For example, the sensor element 101 may have a structure in which two internal cavities, the first internal cavity 20 and the second internal cavity 40 are provided, the inner main pump electrode 22 is disposed in the first internal cavity 20, and the auxiliary pump electrode 51 and the measurement electrode 44 are disposed in the second internal cavity 40. In this case, for example, a porous protective layer covering the measurement electrode 44 may be formed as a diffusion-rate limiting part between the auxiliary pump electrode 51 and the measurement electrode 44.

In the gas sensor 100 of the above embodiment, the outer pump electrode 23 has three functions as an outer main pump electrode in the main pump cell 21, an outer auxiliary pump electrode in the auxiliary pump cell 50, and an outer measurement electrode in the measurement pump cell 41. However, the outer pump electrode 23 is not limited thereto. For example, the outer main pump electrode, the outer auxiliary pump electrode, and the outer measurement electrode may be formed as different electrodes. For example, any one or more of the outer main pump electrode, the outer auxiliary pump electrode, and the outer measurement electrode may be provided on the outer surface of the base part 102 separately from the outer pump electrode 23 so as to be in contact with a measurement-object gas. Alternatively, the reference electrode 42 may also serve as any one or more of the outer main pump electrode, the outer auxiliary pump electrode, and the outer measurement electrode.

### Method for Producing Sensor Element

Next, one example of a method for producing the sensor element as described above is described. A plurality of unfired sheet moldings (so-called green sheets) containing an oxygen-ion-conductive solid electrolyte such as zirconia (ZrO₂) as a ceramic component are subjected to a predetermined processing and printing of circuit pattern, and then the plurality of sheets are laminated, and the laminate was cut, and then fired. Thus the sensor element 101 can be manufactured.

Hereinafter, description is made while taking the case of manufacturing the sensor element 101 composed of six layers shown in FIG. 1 as an example.

First, six green sheets containing an oxygen-ion-conductive solid electrolyte such as zirconia (ZrO₂) as a ceramic component are prepared. For manufacturing of the green sheets, a known molding method can be used. The six green sheets may all have the same thickness, or the thickness differs depending on the layer to be formed. In each of the six green sheets, sheet holes or the like for use in positioning at the time of printing or stacking are formed in advance by a known method such as a punching process with a punching apparatus (blank sheet). In the blank sheet for use as the spacer layer 5, penetrating parts such as internal cavities are also formed in the same manner. Also in the remaining layers, necessary penetrating parts are formed in advance.

The blank sheets for use as six layers, namely, the first substrate layer 1, the second substrate layer 2, the third substrate layer 3, the first solid electrolyte layer 4, the spacer layer 5, and the second solid electrolyte layer 6 are subjected to printing of various patterns required for respective layers and drying treatment. For printing of a pattern, a known screen printing technique can be used. Also as the drying treatment, a known drying means can be used.

In forming the porous coating layer 25, a paste for the porous coating layer 25 is prepared. The paste for the porous coating layer 25 is prepared by blending a raw material powder (in this embodiment, an alumina powder) composed of the material of the porous coating layer 25 as described above, and an organic binder, an organic solvent, etc. A pore forming material for forming pores may be further added. The pore forming material is an organic or inorganic material that will disappear by firing in the subsequent step. Examples of the pore forming material that can be used include a xanthine derivative such as theobromine, an organic resin material such as an acrylic resin, an organic material such as starch, and an inorganic material such as carbon. The paste for the porous coating layer 25 is preferably prepared so that an oxygen diffusion coefficient of the porous coating layer 25 becomes a desired value after firing in the subsequent step. For example, particle size of the raw material powder or a blending ratio of the organic binder may be adjusted so that the oxygen diffusion coefficient of the porous coating layer 25 becomes the desired value. Alternatively, an amount of the pore forming material to be added may be adjusted.

Then, the paste for the porous coating layer 25 is printed and dried in a desired pattern on the printed pattern of the inner main pump electrode 22 that has been printed on the second solid electrolyte layer 6. Also, the paste for the porous coating layer 25 is printed and dried in a desired pattern on the printed pattern of the inner main pump electrode 22 that has been printed on the first solid electrolyte layer 4. The order of these printings may be appropriately determined.

After completing the printing and drying of diverse patterns for each of the six blank sheets by repeating these steps, contact bonding treatment of stacking the six printed blank sheets in a predetermined order while positioning with the sheet holes and the like, and contact bonding at a predetermined temperature and pressure condition to give a laminate is conducted. The contact bonding treatment is conducted by heating and pressurizing with a known laminator such as a hydraulic press. While the temperature, the pressure and the time of heating and pressurizing depend on the laminator being used, they may be appropriately determined to achieve excellent lamination.

The obtained laminate includes a plurality of sensor elements 101. The laminate is cut into units of the sensor element 101. The cut laminate is fired at a predetermined firing temperature to obtain the sensor element 101. The firing temperature may be such a temperature that the solid electrolyte forming the base part 102 of the sensor element 101 is sintered to become a dense product, and an electrode or the like maintains desired porosity. The firing is conducted, for example, at a firing temperature of about 1300 to 1500° C.

The obtained sensor element 101 is incorporated into the gas sensor 100 in such a form that the front end part of the sensor element 101 comes into contact with the measurement-object gas, and the rear end part of the sensor element 101 comes into contact with the reference gas.

### EXAMPLES

Hereinafter, explanation will be made using Examples. The present invention is not limited to the following Examples.

### Examples 1 to 6 and Comparative Example 1

As Examples 1 to 6 and Comparative Example 1, the respective sensor elements were produced as follows, in accordance with the above-described production method of the sensor element 101.

In Example 1, the porous coating layer 25 was in a shape that covered a region of the inner main pump electrode 22 including an electrode end part (an electrode front end part) of the inner main pump electrode 22 closer to the front end part of the base part 102 and having a length of 50% of the length of the inner main pump electrode 22 in the longitudinal direction of the sensor element 101. The oxygen diffusion coefficient of the porous coating layer 25 was 1.7×10⁻⁷ m²/s (this value is referred to as D₁). The thickness of the porous coating layer 25 was 10 μm.

In Example 2, the porous coating layer 25 was in a shape that covered a region of the inner main pump electrode 22 including the electrode end part (the electrode front end part) of the inner main pump electrode 22 closer to the front end part of the base part 102 and having a length of 50% of the length of the inner main pump electrode 22 in the longitudinal direction of the sensor element 101. The oxygen diffusion coefficient of the porous coating layer 25 was 1.7×10⁻⁷ m²/s (D₁). The thickness of the porous coating layer 25 was 20 μm.

In Example 3, the porous coating layer 25 was in a shape that covered a region of the inner main pump electrode 22 including the electrode end part (the electrode front end part) of the inner main pump electrode 22 closer to the front end part of the base part 102 and having a length of 25% of the length of the inner main pump electrode 22 in the longitudinal direction of the sensor element 101. The oxygen diffusion coefficient of the porous coating layer 25 was 1.7×10⁻⁷ m²/s (D₁). The thickness of the porous coating layer 25 was 10 μm.

In Example 4, the porous coating layer 25 was in a shape that covered a region of the inner main pump electrode 22 including the electrode end part (the electrode front end part) of the inner main pump electrode 22 closer to the front end part of the base part 102 and having a length of 50% of the length of the inner main pump electrode 22 in the longitudinal direction of the sensor element 101. The oxygen diffusion coefficient of the porous coating layer 25 was 1.7×10⁻⁷ m²/s (D₁). The thickness of the porous coating layer 25 was 20 μm in an area of a length of 25% of the length of the inner main pump electrode 22 from the electrode front end part, and 10 μm in the remaining area.

In Example 5, the porous coating layer 25 was in a shape that covered a region of the inner main pump electrode 22 including the electrode end part (the electrode front end part) of the inner main pump electrode 22 closer to the front end part of the base part 102 and having a length of 50% of the length of the inner main pump electrode 22 in the longitudinal direction of the sensor element 101. The oxygen diffusion coefficient of the porous coating layer 25 was 1.7×10⁶ m²/s (10D₁). The thickness of the porous coating layer 25 was 10 μm.

In Example 6, the porous coating layer 25 was formed to cover the entire inner main pump electrode 22. The inner main pump electrode 22 was divided into four areas in the longitudinal direction of the sensor element 101, and oxygen diffusion coefficients of the porous coating layer 25 on the respective areas were set to different values. The oxygen diffusion coefficients of the four areas were 6.8×10⁻⁸ m²/s (0.4D₁), 1.7×10⁻⁷ m²/s (D₁), 1.7×10⁻⁶ m²/s (10D₁), and 1.36×10⁵ m²/s (80D₁), respectively, from the electrode front end part of the inner main pump electrode 22 toward the rear end of the sensor element 101. The thickness of the porous coating layer 25 was 10 μm.

Comparative Example 1 was configured without the porous coating layer 25 as a conventional example.

In each of Examples 1 to 6, the ceiling electrode portion 22a and the bottom electrode portion 22b in the inner main pump electrode 22 were the same in size. The ceiling coating layer 25a and the bottom coating layer 25b in the porous coating layer 25 were formed in the same size on the ceiling electrode portion 22a and the bottom electrode portion 22b, respectively.

In each of Examples 1 to 6, the porous coating layer 25 had a shape that was longer by the same length as the thickness of the porous coating layer 25 toward the front end side of the sensor element 101 from the electrode front end of the inner main pump electrode 22. The porous coating layer 25 also had a shape that was longer by the same length as the thickness of the porous coating layer 25 from each of the electrode ends in the width direction of the inner main pump electrode 22.

In each of Examples 1 to 6 and Comparative Example 1, the size of the first internal cavity 20 was 3.3 mm in length in the longitudinal direction of the sensor element 101, 2.5 mm in width and 100 μm in thickness perpendicular to the longitudinal direction of the sensor element 101. The size of each of the ceiling electrode portion 22a and the bottom electrode portion 22b was 3.1 mm in length in the longitudinal direction of the sensor element 101, 2.3 mm in width and 10 μm in thickness perpendicular to the longitudinal direction of the sensor element 101.

### Evaluation of Maximum Current Density in Pump Current Flowing Through Main Pump Cell

For each of the above-described sensor elements, maximum current density of the pump current Ip0 flowing through the main pump cell 21 was evaluated. Current density distribution of the pump current Ip0 flowing through the main pump cell 21 in the inner main pump electrode 22 was obtained. A measurement-object gas atmosphere was NO=500 ppm and O₂=5% (the remainder is N₂). For each of the ceiling electrode portion 22a and the bottom electrode portion 22b of the inner main pump electrode 22, the length in the longitudinal direction was divided into segments of 100 μm each, and the current density in each segment was calculated to obtain a value of the maximum current density.

In this Examples, results of Examples 3, 4, and 6 were based on a simulation. It is to be noted that the measurement results of Examples 1, 2, 5 and Comparative Example 1 corresponded well with the simulation results. That is, the order of the mitigating effect on the current concentration predicted from the measurement results of the experiment matched well with the order of the mitigating effect on the current concentration calculated by the simulation.

In all of the sensor elements, maximum current density Jmax was provided at the electrode front end part (in this evaluation, an area having a length of 100 μm in the longitudinal direction of the sensor element 101 from the electrode front end) of the ceiling electrode portion 22a in the inner main pump electrode 22.

In each of Examples 1 to 6 and Comparative Example 1, a current density ratio rⱼ of the maximum current density Jmax to the maximum current density Jmax in Comparative Example 1 was obtained. The current density ratio rⱼ smaller than 1 indicates that the maximum current density Jmax is smaller than that in Comparative Example 1. That is, the current density ratio rⱼ smaller than 1 indicates that the current concentration on the electrode front end of the inner main pump electrode 22 is mitigated. The smaller the current density ratio rⱼ, the more the current concentration is mitigated.

An improving effect on the current concentration in each of Examples 1 to 6 and Comparative Example 1 was evaluated according to the following criteria based on a value of the current density ratio rⱼ.
A: Current density ratio rⱼ is less than 0.8;
B: Current density ratio rⱼ is not less than 0.8 and less than 1.0; and
C: Current density ratio rⱼ is not less than 1.0.

When the evaluation is A or B, it is indicated that the current concentration is mitigated.

Table 1 shows:
an area ratio (%) of the part covered by the porous coating layer 25 in the inner main pump electrode 22,
the oxygen diffusion coefficient (m²/s) of the porous coating layer 25,
the thickness (μm) of the porous coating layer 25, and
the evaluation result of the improving effect on the current concentration, in each of Examples 1 to 6 and Comparative Example 1.

TABLE 1

| | Porous coating layer 25 | | | |
|---|---|---|---|---|
| | | Oxygen | | Improving |
| | Area | diffusion | | effect |
| | ratio | coefficient | Thickness | on current |
| | (%) | (m²/s) | (μm) | concentration |
| Example 1 | 50 | 1.7 × 10⁻⁷ | 10 | A |
| Example 2 | 50 | 1.7 × 10⁻⁷ | 20 | A |
| Example 3 | 25 | 1.7 × 10⁻⁷ | 10 | B |
| Example 4 | 50 | 1.7 × 10⁻⁷ | 25% from front | A |
| | | | end: 20 | |
| | | | reminder part: | |
| | | | 10 | |
| Example 5 | 50 | 1.7 × 10⁻⁶ | 10 | C |
| Example 6 | 100 | 6.8 × 10⁻⁸ | 10 | A |
| | | 1.7 × 10⁻⁷ | | |
| | | 1.7 × 10⁻⁶ | | |
| | | 1.36 × 10⁻⁵ | | |
| | | for 25% each | | |
| | | from front end | | |
| Comparative | — | — | — | C |
| Example 1 | | | | |

It was confirmed that the current concentration on the electrode front end part in the inner main pump electrode 22 was mitigated in all of Examples 1 to 4, and 6 compared to Comparative Example 1.

In Example 5, the improving effect on the current concentration was not observed in comparison to Comparative Example 1. However, since the porous coating layer 25 is present, it is considered to be possible to effectively suppress Au evaporation from a region of the inner main pump electrode 22 from which Au likely to evaporate. This effect is considered to make it possible to effectively suppress the deterioration of the NOx decomposition activity in the measurement electrode 44. It is also considered that NOx decomposition in the inner main pump electrode 22 can be more suppressed. As a result, the effect is expected to suppress decrease in detection accuracy due to long-term use of the gas sensor.

### Durability Test

Gas sensors provided with the respective sensor elements of Examples 1 to 2 and Comparative Example 1 among the above-described sensor elements were subjected to a durability test in the air, and the linearity R² (coefficient of determination R²) of NOx output with respect to oxygen concentration was evaluated for each of the gas sensors. As described above, by using this linearity R² (coefficient of determination R²) of NOx output with respect to oxygen concentration, the degree of NOx decomposition in the inner main pump electrode 22 can be evaluated. Specifically, evaluation is done in the following manner.

First, new gas sensors of Examples 1 to 2 and Comparative Example 1 were measured in a model gas device, respectively. Each of the gas sensors was attached to a piping for measurement of the model gas device, and was driven (a driving temperature was about 850° C.). A model gas satisfying NO=500 ppm and O₂=0% was flowed in the piping for measurement, and a Ip2 current value (Ip2_{(500.0)} of each of the gas sensors was measured. Also for the model gas satisfying NO=500 ppm and O₂=5%, the model gas satisfying NO=500 ppm and O₂=10%, and the model gas satisfying NO=500 ppm and O₂=18%, Ip2 current values (Ip2_{(500.5)}, Ip2_{(500.10)}, Ip2_{(500.18)}) of each of the gas sensors were respectively measured in the same manner. The gas component other than NO and O₂ in the model gas used for measurement was N₂ (remainder).

The coefficient of determination R² was calculated in the linear regression equation between the oxygen concentrations of the model gas, and measured four Ip2 values (Ip2_{(500.0)}, Ip2_{(500.5)}, Ip2_{(500.10)}, Ip2_{(500.18)}).

Next, the durability test was conducted for each of the gas sensors. Specifically, each of the gas sensors was driven in the air (the driving temperature was about 850° C.), and a continuous operating test (an air continuous test) was performed for 1500 hours. At the point of time after a lapse of 500 hours from the start of the test, the durability test was suspended, and the coefficient of determination R² after a lapse of 500 hours of the durability test was calculated in the method described above. Then, the durability test was resumed, and the coefficient of determination R² was calculated in the same manner at each of the point of time after a lapse of 1000 hours and the point of time after a lapse of 1500 hours from the start of the test.

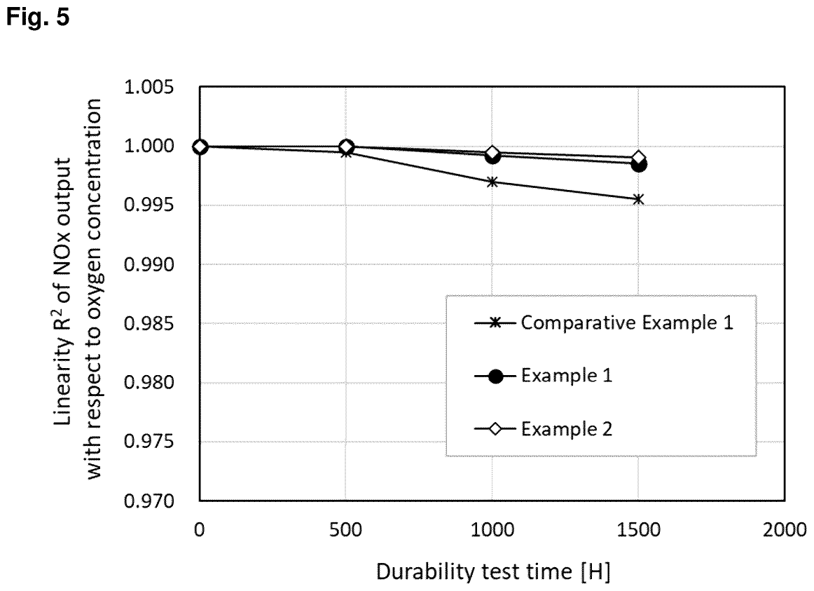

Table 2 and FIG. 5 show the durability test results of Examples 1 to 2 and Comparative Example 1. In FIG. 5, the vertical axis of the graph represents the linearity R² (coefficient of determination R²) of NOx output with respect to oxygen concentration, and the horizontal axis represents the durability time (H: hours).

TABLE 2

| | Porous coating layer 25 | | | Linearity R² of NOx output | | | |
|---|---|---|---|---|---|---|---|
| | | Oxygen | | with respect to oxygen | | | |
| | Area | diffusion | | concentration in durability test | | | |
| | ratio | coefficient | Thickness | 0 | 500 | 1000 | 1500 |
| | (%) | (m²/s) | (μm) | hour | hours | hours | hours |
| Example 1 | 50 | 1.7 × 10⁻⁷ | 10 | 1.0000 | 1.0000 | 0.9992 | 0.9985 |
| Example 2 | 50 | 1.7 × 10⁻⁷ | 20 | 1.0000 | 1.0000 | 0.9995 | 0.9990 |
| Comparative | — | — | — | 1.0000 | 0.9995 | 0.9970 | 0.9955 |
| Example 1 | | | | | | | |

As shown in Table 2 and FIG. 5, it was confirmed that the linearity R² (coefficient of determination R²) of NOx output with respect to oxygen concentration in each of Examples 1 to 2 was maintained higher after the durability test in comparison with Comparative Example 1. That is, it was confirmed that the decrease in the coefficient of determination R² was more suppressed in Examples 1 to 2 than in Comparative Example 1. It is considered that due to the effect of mitigating the current concentration as described above, the NOx decomposition in the inner main pump electrode 22 was more suppressed even after the durability test.

As described above, according to the present invention, since evaporation of Au from the inner main pump electrode 22 can be suppressed, attachment of Au to the measurement electrode 44 can be suppressed. Further, according to the present invention, since the current concentration at the position close to the gas inlet 10 in the inner main pump electrode 22 can be mitigated, NOx decomposition in the inner main pump electrode 22 can be further suppressed even when a resistance value of the main pump cell 21 is increased due to long-term use of the gas sensor 100. As a result, the decrease in the detection accuracy due to the long-term use of the gas sensor can be suppressed.

### EXPLANATION OF REFERENCE SIGNS IN THE DRAWINGS

1: first substrate layer; 2: second substrate layer; 3: third substrate layer; 4: first solid electrolyte layer; 5: spacer layer; 6: second solid electrolyte layer; 10: gas inlet; 11: first diffusion-rate limiting part; 12: buffer space; 13: second diffusion-rate limiting part; 15: measurement-object gas flow part; 20: first internal cavity; 21: main pump cell; 22: inner main pump electrode; 22a: ceiling electrode portion (of the inner main pump electrode); 22b: bottom electrode portion (of the inner main pump electrode); 23: outer pump electrode; 24: variable power supply (of the main pump cell); 25: porous coating layer; 25a: ceiling coating layer (of the porous coating layer); 25b: bottom coating layer (of the porous coating layer); 30: third diffusion-rate limiting part; 40: second internal cavity; 41: current measurement pump cell; 42: reference electrode; 43: reference gas introduction space; 44: measurement electrode; 46: variable power supply (of the current measurement pump cell); 48: air introduction layer; 50: auxiliary pump cell; 51: auxiliary pump electrode; 51a: ceiling electrode portion (of the auxiliary pump electrode); 51b: bottom electrode portion (of the auxiliary pump electrode); 52: variable power supply (of the auxiliary pump cell); 60: fourth diffusion-rate limiting part; 61: third internal cavity; 70: heater part; 71: heater electrode; 72: heater; 73 through hole; 74 heater insulating layer; 75: pressure relief vent; 76: heater lead; 80: oxygen-partial-pressure detection sensor cell for main pump control; 81: oxygen-partial-pressure detection sensor cell for auxiliary pump control; 82: oxygen-partial-pressure detection sensor cell for measurement pump control; 83: sensor cell; 100: gas sensor; 101: sensor element; and 102: base part.

What is claimed is:

1. A sensor element for detecting a target gas to be measured in a measurement-object gas, the sensor element comprising:
a base part in an elongated plate shape, including an oxygen-ion-conductive solid electrolyte layer;
a measurement-object gas flow part formed from one end part in a longitudinal direction of the base part;
an inner main pump electrode disposed on an inner surface of the measurement-object gas flow part;
a porous coating layer covering at least an electrode end part of the inner main pump electrode closer to the one end part in the longitudinal direction of the base part; and
a measurement electrode disposed at a position farther from the one end part in the longitudinal direction of the base part than the inner main pump electrode on the inner surface of the measurement-object gas flow part.

2. The sensor element according to claim 1, wherein the porous coating layer covers 3% or more of area of the inner main pump electrode.

3. The sensor element according to claim 1, wherein an oxygen diffusion coefficient of the porous coating layer is 1×10⁻⁶ m²/s or less in at least a part of the porous coating layer.

4. The sensor element according to claim 1, wherein a thickness of the porous coating layer is 1 μm or more.

5. The sensor element according to claim 1, wherein an oxygen diffusion coefficient of the porous coating layer varies in the longitudinal direction of the base part.

6. The sensor element according to claim 1, wherein an oxygen diffusion coefficient of the porous coating layer is stepwise or continuously increased from a closer side toward a farther side with respect of the one end part in the longitudinal direction of the base part.
